SEQUENCE LISTING (6 sequences)

SEQ ID NO: 1 - DNA, 19 bp, Artificial sequence
  5′ primer
gctctttgggagaggtggg

SEQ ID NO: 2 - DNA, 21 bp, Artificial sequence
  3′ primer
tgggttttcagtgaacgtggt

SEQ ID NO: 3 - DNA, 16 bp, Artificial sequence
  Forward primer (Vhbio Ltd, Gateshead, UK)
tgctgcccctgctgct

SEQ ID NO: 4 - DNA, 19 bp, Artificial sequence
  Reverse primer (Vhbio Ltd, Gateshead, UK)
acctcaaggaccagctcgg

SEQ ID NO: 5 - DNA, 16 bp, Articial sequence
  FAM probe (allele 2) (Applied Biosystems, UK)
agatgagcccccagaa

SEQ ID NO: 6 - DNA, 17 bp, Artificial sequence
  VIC probe (allele 1) (Applied Biosystems, UK)
cagatgatcccccagaa

(19) United States
(12) Patent Application Publication
(10) Pub. No.: US 2011/0027785 A1
(43) Pub. Date: Feb. 3, 2011

(54) METHOD FOR DETERMINING SUSCEPTIBILITY OF INDIVIDUALS TO POLYPHENOLS

(75) Inventor: Anna Louise Brown, Sharnbrook (GB)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/825,376

(22) Filed: Jun. 29, 2010

(30) Foreign Application Priority Data
Jun. 30, 2009 (EP) EP09164238

(51) Int. Cl. C12Q 1/68; G01N 33/573

(52) U.S. Cl. 435/6; 435/7.4

(57) ABSTRACT

Consumption of high energy diets, lack of physical activity and sleep curtailment have all been linked to the development of type II diabetes and cardiovascular disease. Studies have also shown how powerful diet and lifestyle modification can be for preventing disease. However not everyone responds to lifestyle change in the same way. This is because genetic factors can modify biological response to environmental challenge.

This invention relates to a method for determining the predisposition of an individual to treatment to alleviate or pre-empt a particular medical condition. In particular to a method for determining the predisposition of an individual to epigallocatechin gallate, catechin, gallocatechin, catechin gallate, gallocatechin gallate, epicatechin, epigallocatechin, epicatechin gallate and mixtures thereof for the treatment and/or prevention of at least one of:
(a) high diastolic blood pressure;
(b) type II diabetes; and
(c) vascular stiffness.

This invention relates to a method for determining the predisposition of an individual to treatment to alleviate or pre-empt particular medical conditions. In particular to a method for determining the predisposition of an individual to epigallocatechin gallate, catechin, gallocatechin, catechin gallate, gallocatechin gallate, epicatechin, epigallocatechin, epicatechin gallate and mixtures thereof for the treatment and/or prevention of at least one of:

(a) high diastolic blood pressure;
(b) type II diabetes; and
(c) vascular stiffness.

It has long been recognised that diet and lifestyle factors play an important role in health as well as disease. Consumption of high energy diets, lack of physical activity and sleep curtailment have all been linked to the development of type II diabetes and cardiovascular disease. Conversely studies have also shown how powerful diet and lifestyle modification can be for preventing disease, particularly in high risk individuals. However not everyone responds to lifestyle change in the same way. This is because genetic factors can modify biological response to environmental challenge, in other words genetic, dietary and environmental factors interact and these interactions may influence health.

Genetic polymorphisms represent the differences in deoxyribonucleic acid (DNA) sequences between individuals. Although 99.9% of human DNA sequences are identical, the 0.1% difference between individuals can have significant biological effects. Indeed genetic factors which influence the absorption, metabolism or transport of nutrients will modify the way in which an individual responds to a particular diet, potentially affecting disease susceptibility and/or trajectory.

In particular the health effects of dietary flavonoids appear to be influenced by variability in flavonoid O-methylation, a major pathway of flavonoid metabolism catalysed by the enzyme catechol-O-methyltransferase (COMT). The general function of COMT is to eliminate potentially active or toxic catechol-based compounds from the body. A common genetic polymorphism (the single nucleotide polymorphism (SNP) between guanine and adenine in the COMT gene in rs4680) has been identified that alters the function of the COMT enzyme. This polymorphism, which results in an amino acid (valine to methionine) substitution, has been shown to reduce the thermostability of the enzyme and is associated with 3-4 fold lower enzyme activity. Individuals who are heterozygous at this genetic position and are therefore capable of expressing both the high and low activity form of the enzyme have O-methyl transferase activity intermediate to the two homozygous conditions. Given the effect of the valine/methionine polymorphism on thermostability and enzyme activity, it is likely that COMT genotype will influence the rate of catechin metabolism which may in turn influence the functional response to dietary catechins such as epigallocatechin gallate (EGCG).

### SUMMARY OF THE INVENTION

The invention provides a method for determining the predisposition of an individual to epigallocatechin gallate, catechin, gallocatechin, catechin gallate, gallocatechin gallate, epicatechin, epigallocatechin, epicatechin gallate and mixtures thereof for the treatment and/or prevention of at least one of:

(a) high diastolic blood pressure;
(b) type II diabetes; and
(c) vascular stiffness;
the method comprising the steps of:
(i) obtaining an ex-vivo sample of an individual;
(j) determining the catechol-O-methyltransferase genotype of the individual from the sample or the catechol-O-methyltransferase activity of the sample;
(k) determining that when the individual has an adenine-adenine or adenine-guanine catechol-O-methyltransferase genotype or a lowest quartile of catechol-O-methyltransferase activity,
(A) they are more sensitive to treatment by epigallocatechin gallate for the treatment and/or prevention of high diastolic blood pressure than an individual with a guanine-guanine catechol-O-methyltransferase genotype or a highest quartile of catechol-O-methyltransferase activity;
(B) they are more sensitive to treatment by epigallocatechin gallate for the treatment and/or prevention of type II diabetes than an individual with a guanine-guanine catechol-O-methyltransferase genotype or a highest quartile of catechol-O-methyltransferase activity; and
(C) they are less sensitive to treatment by epigallocatechin gallate for the treatment and/or prevention of vascular stiffness than an individual with a guanine-guanine catechol-O-methyltransferase genotype or a highest quartile of catechol-O-methyltransferase activity.

Preferably the predisposition of an individual is to epigallocatechin gallate.

The step of determining the catechol-O-methyltransferase genotype of the individual may comprise the step of extracting genomic deoxyribonucleic acid from the sample.

The step of determining the catechol-O-methyltransferase activity of the sample may comprise the steps of extracting a protein fraction from the sample, the protein fraction comprising catechol-O-methyltransferase, and then contacting the protein fraction with a substrate which would indicate catechol-O-methyltransferase activity.

When the ex-vivo sample is urine, the step of determining the catechol-O-methyltransferase activity of the sample comprises the step of determining the level of species selected from the group consisting of catechol-O-methyltransferase substrate, methylated catechol-O-methyltransferase substrate, downstream metabolites of methylated catechol-O-methyltransferase substrate and mixtures thereof.

### BRIEF DESCRIPTION OF THE FIGURES

The invention is now exemplified with reference to the following figures which show in:

FIG. 1a diastolic blood pressure (mm Hg) for EGCG treatment group for guanine-guanine COMT genotype group; and

FIG. 1b diastolic blood pressure (mm Hg) for EGCG treatment group for adenine-guanine COMT genotype group; and

FIG. 1c diastolic blood pressure (mm Hg) for EGCG treatment group for adenine-adenine COMT genotype group; and

FIG. 2 LSMean change from baseline (mmHG) for EGCG treatment group for guanine-guanine, adenine-guanine and adenine-adenine COMT genotype groups; and

FIG. 3 the calculation of vascular stiffness (SI_{DVP}) from digital volume pulse measurements.

### DETAILED DESCRIPTION OF THE INVENTION

### 1. Diastolic Blood Pressure

### 1.1 Study Design

This was a double blind, single centre, randomised and parallel design study. A total of 95 male participants were recruited on to the study using the following criteria:
healthy (no significant history or current disease or medication)
non-diabetic
non-smokers
aged 40-65 years
Body Mass Index >28 <38

The study population were randomly assigned to one of two treatment groups. Each group was balanced for age and insulin resistance (homeostatic model of insulin resistance HOMAᵢᵣ). HOMAᵢᵣ was measured from mean fasting plasma glucose values as described by Mathews et al, Diabetologia (1985), 28, 412-419. 75 subjects consented to genotype analysis for the COMT polymorphism rs4680.

The active treatment was 800 mg/day EGCG and the placebo treatment 800 mg/day lactose. Treatments were administered twice daily with food for 8 weeks, one 400 mg capsule in the morning and one 400 mg capsule in the evening. The diastolic blood pressure was measured pre- and post-treatment. During the three days before pre- and post-treatment study visits, participants refrained from exercise, alcohol and ate their normal diet which consisted of at least 150 g carbohydrates.

### 1.2 COMT genotyping

Genomic DNA was extracted from whole blood samples (1 ml) using an Agowa magnetic Maxi DNA isolation kit on an automated platform (Hamilton Star) according to manufacturer's instructions. 50 ng of purified genomic DNA was subjected to polymerase chain reaction (PCR) amplification in 50 μl of 1×PCR buffer (ABgene), 20 mM deoxynucleotide triphosphates (dNTPs), 25 pmoles 5′ primer (GCTCTTTGGGAGAGGTGGG), 25 pmoles 3′ primer (TGGGTTTTCAGTGAACGTGGT), 2.5 units Thermo-Start DNA polymerase. Cycling conditions were 30 cycles of 94° C. for 15 seconds, 55° C. for 15 seconds and 72° C. for 120 seconds using a Perkin Elmer PCR machine. The initial denaturing step (94° C.) was extended to 15 minutes. The PCR fragments were then purified using a MinElute 96 UF PCR Purification kit according to the manufacturer's instructions. 10 ng of each purified PCR fragment was then sequenced using an ABI PRISM 2′-deoxyguanosine 5′-triphosphate (dGTP) BigDye terminator cycle sequencing kit. Unincorporated fluorescent nucleotides were removed using Agencourt's CleanSeq SPRI magnetic clean up kit according to the manufacturer's instructions. The sequencing reaction was then separated on an ABI 3730 DNA sequencer before analysis of the data on Lasergenes DNAStar Seqman II software package.

### 1.3 Diastolic Blood Pressure Measurements

Blood pressure was measured manually on the upper arm using a sphygmomanometer (UA-787, A and D Medical). Three measurements were taken at 5 minute intervals whilst participants rested in a semi-recumbent position and with participants rested for at least 5 minutes before the first measurement. All three measurements were used to derive mean blood pressure values. Non-smoking status and alcohol abstinence were verified using Micro CO meter (Micro Medical Ltd) and AlcoMate Pro (AK Solutions) monitors, respectively. All equipment was calibrated before use.

### 1.4 Statistical Analyses

The data was stratified based on the derived COMT genotype and analysed. For each treatment group and genotype subset the mean and standard deviation for the change from baseline to endpoint was calculated. Paired t-tests were used to assess whether the change from baseline was statistically significant (p<0.05). Baseline values were included as a covariates in the final model and a t-test performed to test whether the LSMean of the analysis variable (i.e. change from baseline) for each genotype group was significantly different (p<0.05) from zero.

### 1.5 Results

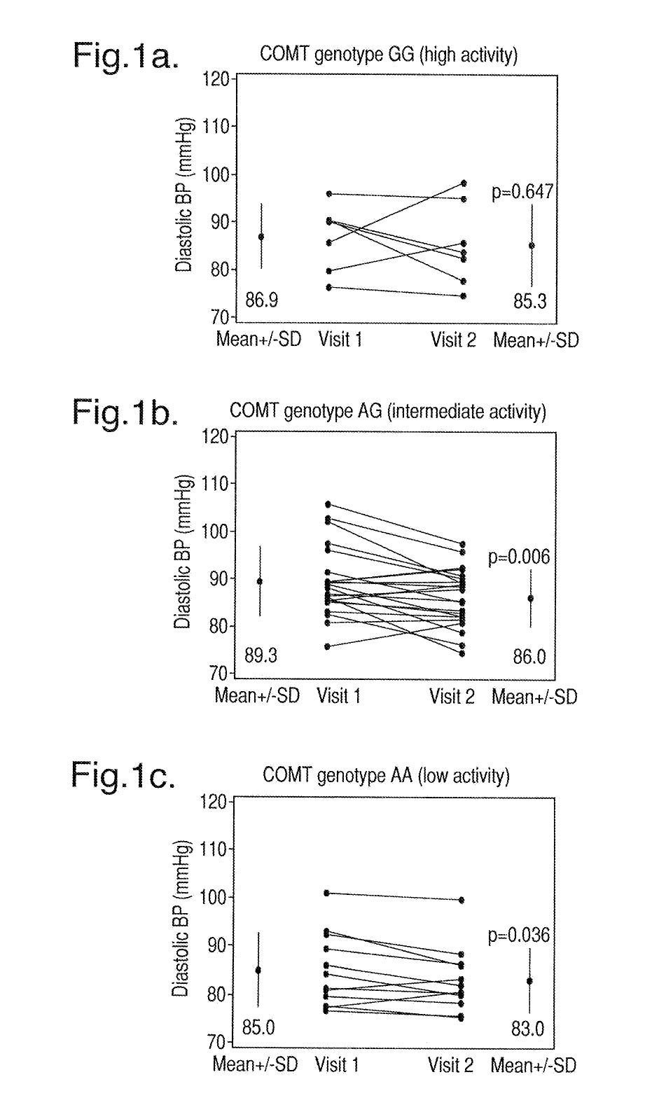

FIGS. 1a, 1b and 1c show the diastolic blood pressure data for each of the individuals in the EGCG intervention group. Calculation of the means and standard deviations for each of the sub-groups revealed a statistically significant reduction (compared to baseline) in diastolic blood pressure for the adenine-adenine (AA) (low activity; p=0.036) and adenine-guanine (AG) (intermediate activity; p=0.006) forms of COMT. Although the guanine-guanine (GG) (high activity) group also showed a mean reduction in diastolic blood pressure, this result was not statistically significant (p=0.647).

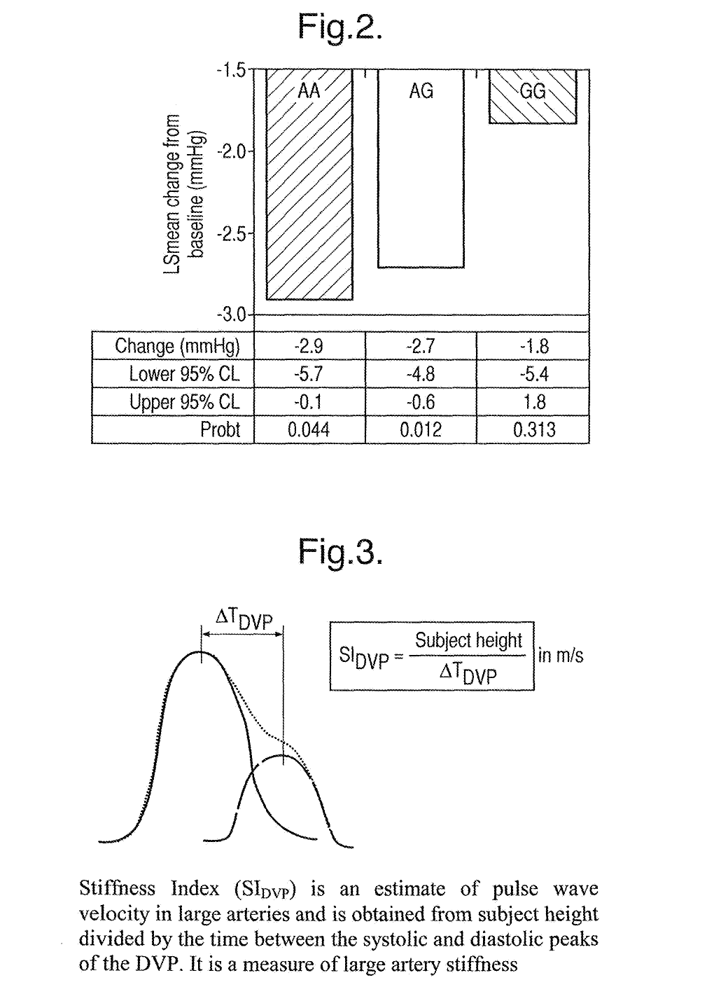

FIG. 2 shows the results of further analyses which controlled for baseline diastolic blood pressure and confirmed that the EGCG intervention resulted in a statistically significant reduction in blood pressure (change from baseline) in both the AA (p=0.044) and AG (p=0.012) groups but not the GG group.

No statistically significant differences in diastolic blood pressure measurements were found in the placebo group.

### 1.6 Discussion

From the population stratification performed (based on COMT genotype) differences can be seen in the magnitude of effect from the EGCG supplementation. Statistically significant reductions from baseline in diastolic blood pressure were found in the AA and AG groups but not in the GG group. The greatest reduction was seen for the group carrying the lowest activity variant of COMT. The group carrying the highest activity form of the enzyme had the least benefit from the EGCG intervention, although it should be remembered that this group did still show a reduction in diastolic blood pressure.

### 2. Type II Diabetes and Vascular Stiffness

### 2.1 Study Design

This was a non-placebo controlled single centre parallel design study with two groups; subjects with a homozygous low activity COMT genotype (AA) and subjects with a homozygous high activity COMT genotype (GG).

The subjects were recruited from the Hugh Sinclair and Sensory Dimensions databases and via poster and leaflet advertising. The subjects were asked to provide a fasting blood sample and height, weight, waist circumference, hip circumference and blood pressure measured were made to assess eligibility of entry. The collected blood sample was used to assess liver function together with haematological analysis, cholesterol and triglyceride levels. Individuals with a total-cholesterol >8.0 mmol/l, BMI >35 or blood pressure >160/100 mmHg were not recruited onto the study and advised to consult their GP. All biochemical measurements were obtained by iLab 600 colorimetric analysis (Clinical Chemistry System, Instrumentation Laboratory, Italy) aside from haemoglobin measurements which were performed at The Royal Berkshire Hospital, Reading. Genetic profiling of COMT in addition to the endothelial nitric oxide synthase (eNOS (G298T)) genotype (a polymorphism known to influence vascular reactivity) was carried out on purified genomic DNA to assess eligibility and to assign grouping, with equal numbers of the eNOS genotype distributed within the COMT subgroups. As vascular reactivity is dependent on age, BMI and gender, groups were stratified for such characteristics.

20 subjects were recruited for this study, 10 subjects of each homozygous COMT genotype according to the inclusion and exclusion criteria set forth hereinbelow:

Inclusion criteria
BMI 25-35 and waist circumference >94 cm for men and >80 cm for women
Men and postmenopausal women
Over 18 and under 70 years old
COMT homozygous G or A carriers
Non-smoker
Clinical chemistry parameters within the normal reference range

### Exclusion Criteria

Regular green tea consumers
Non-black tea consumers
On a weight reducing dietary regimen
Taking cholesterol-lowering or blood-pressure reducing medication
Suffering from any form of known gastrointestinal disorders, liver disease, kidney disease or diabetes mellitus
Suffered a myocardial infarction or stroke within the last 12 months
Elevated liver enzymes

Subjects with a BMI in the range of 25-35 and waist circumference of >94 cm for males and >80 cm for females were recruited as there is evidence to suggest subjects within this population group have impaired vascular function. It is thought that habitual consumption of tea could induce an adaptive response affecting metabolism of tea catechins. To reduce the variability in response only regular tea drinkers were included.

Subjects were requested to refrain from intensive exercise, alcohol, high catechol-containing flavonoid food and beverages (such as tea, coffee, chocolate, onions and fruit juice) and dietary supplements for 24 hrs before the study day. A standardised meal was supplied for the evening meal prior to each visit.

1.1 g Sunphenon 90LB (Taiyo, Japan), a commercially available decaffeinated green tea extract containing 880 mg (>80%) green tea catechins, was used as the active treatment for both study groups. The green tea extract was packaged by DHP (Wales, UK) in size 00 vegetarian hydroxypropyl methylcellulose capsules. A chemical analysis of each capsule is given in table 1 hereinbelow.

Supplementation occurred after a 12 hour fast (t=0 h) and subjects remained fasted for 1 hour post treatment because it is thought that the oral bioavailability of EGCG is greater in the fasted compared to the fed state. Ad lib mineral water containing low nitrite levels was supplied during the 8 hour test period.

TABLE 1
Chemical analysis of each capsule.
Sunphenon 90LB

| Chemical | Analysis | Capsule content | |
|---|---|---|---|
| Total polyphenols | 99.97% | 530 | mg |
| Total catechin | 82.71% | 440 | mg |
| EGCG | 43.92% | 232 | mg |
| Epigallocatechin (EGC) | 19.7% | 104 | mg |
| Epicatechin (EC) | 9.5% | 50.3 | mg |
| Caffeine | 0.92% | 4.88 | mg |

A low-flavonoid standardised cereal breakfast was given 1 hour after administration of the green tea catechin supplement. A standardised lunch was given 4 hours after supplementation, consisting of a white bread, soft cheese, a cucumber sandwich, crisps and shortbread biscuits.

Blood samples were obtained by cannulation. Serum separation (1×5 ml), heparin (1×10 ml) and EDTA (1×10 ml) vacutainers were used for time points 0, 2, 3, 4, 6 and 8 hours and EDTA vacutainers only (1×10 ml) for time points 0.5, 1, 1.5 and 2.5 hours. The day plan is set forth hereinbelow:

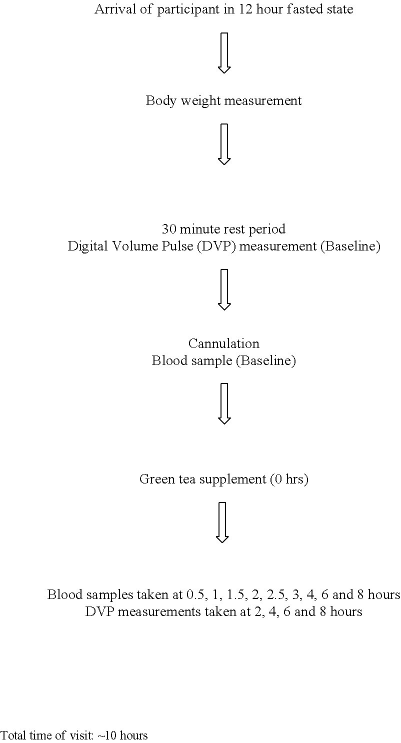

### 2.2 COMT and eNOS Genotyping

### Genomic DNA Isolation

A QIAamp DNA Mini Kit (Qiagen Ltd, UK) was used for DNA purification in accordance with the manufacturer's instructions.

### COMT Genotyping Assay

Mastermix multiplied for number of samples plus 3

12.5 μL TaqMan Universal PCR Mastermix (Applied Biosystems, UK)

1.25 μL 20× TaqMan Drug Metabolism Genotyping Assay Mix (Applied Biosystems, UK)

6.25 μL RNAse/DNAse free water

eNOS Genotyping Assay

Mastermix multiplied for number of samples plus 3

```
12.5 μL TaqMan Universal PCR Mastermix (Bio-
systems, UK)
  1.13 μL Forward primer
TGCTGCCCCTGCTGCT (Vhbio Ltd, Gateshead, UK)
  1.13 μL Reverse primer
ACCTCAAGGACCAGCTCGG (Vhbio Ltd, Gateshead, UK)
  0.33 μL FAM probe (allele 2)
AGATGAGCCCCCAGAA (Applied Biosystems, UK)
  0.33 μL VIC probe (allele 1)
CAGATGATCCCCCAGAA (Applied Biosystems, UK)
  4.58 μL RNAse/DNAse free water
```

### Genotyping Using Real Time (RT)-PCR

COMT/eNOS mastermix was vortexed for 3 seconds and centrifuged at 2500 rpm for 2 minutes (Genofuge 16M Techne, Staffordshire, UK);
20 μL of this solution was pipetted into separate wells of a 96 well RT-PCR plate;
5 μL of the previously isolated DNA was added to the appropriate wells;
5 μL DNA controls for all three possible genotypes were also added to the plate to ease later allele assignment. 3 blank wells containing RNAse/DNAse free water were also included as a background measure;
The plate was subsequently covered (Abgene QPCR optical seal AB-1170, Epsom, UK) and centrifuged at 1000 rpm for 2 minutes (Allegra 6R Centrifuge, Beckman Coulter, Bucks, UK) to remove any air bubbles;
7300 RT PCR system (Applied Biosystems, CA, USA) was used;
The plate was pre-read followed by PCR cycling reactions: 55 C for 0 minutes, 95 C for 10 minutes (1 cycle); 92 C for 15 seconds and 55 C for 90 seconds (1 min for eNOS) (50 cycles);
The plate was post-read and allelic discrimination software used to assign allele 1, allele 2 or both to each sample.

### 2.3 Clinical Chemistry Analysis

Blood samples were collected in 5 ml serum separation, 10 ml EDTA and 10 ml Heparin vacutainers (BD Vacutainer Systems, Plymouth, UK). Samples were kept on ice and processed within two hours of collection. After centrifugation at 3000 rpm for 10 minutes (Heraeus Megafuge 1.0R, Kendro), plasma was aliquoted into cryovials (Greiner bio-one, Germany) in 500 μL quantities. 10 μL of vitamin C buffer was added to the plasma samples to be analysed by high performance liquid chromatography for EGCG measurement to prevent oxidisation and degradation of EGCG. Samples were stored at −80° C. until analysis.

Samples were analysed for the following:

| | | |
|---|---|---|
| Liver enzymes | | |
| Cholesterol | | |
| Haematological | | all measured using |
| analysis | {close oversize brace} | colorimetric analysis (iLab) |
| Triglycerides | | |
| Glucose | | |

Insulin measured using a commercially available ELISA kit

iLab 600 Colourimetric Analysis Procedure

Quantitative in vitro diagnostic determination of plasma triglycerides, total cholesterol and liver enzymes (alanine aminotransferase (ALT), γ-glutamyl transferase (γ-GT) and bilirubin) was completed using iLab 600. 160 μL of plasma was transferred to an appropriately labelled 3 ml polystyrene cup (LP Italiana Spa, Italy). SeraChem Level 1 and SeraChem Level 2 (Instrumentation Laboratory, Italy) were used as quality controls. The variability of repeated measures for the quality controls was also carried out with all calculated variations being within the acceptable range (coefficient of variation <3%). Reagents Triglycerides R2 and Cholesterol R1 (Clinical Chemistry System, Instrumentation Laboratory, Italy) provided the necessary enzymes, cofactors, stabilisers and buffers needed for efficient quantification. ReferrIL G Calibrator (Clinical Chemistry System, Instrumentation Laboratory, Italy) was used to recalibrate the instrument after each reagent addition.

### Glucose Test:

Glucose was measured using bichromatic analysis and hexokinase methodology via the following reaction:

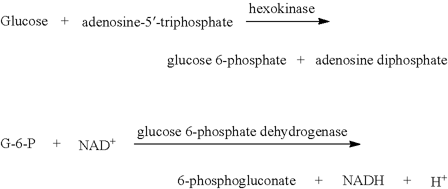

Absorbance measurements taken at wavelength 340 nm and blanking wavelength 375 nm are directly proportional to the glucose in the plasma sample.

The remaining iLab tests were carried out using assays known to the person skilled in the art.

### 2.4 Insulin Quantification

Quantification of insulin in the plasma samples collected in this study was determined by an enzyme immunoassay kit (DakoCytomation, Cambridgeshire, UK). The assay uses a sandwich enzyme immunoassay technique. The microplate is coated with a specific anti-insulin antibody and when incubated with the sample/control and enzyme-labelled antibody a complex is formed. Washing removes unbound enzyme-labelled antibody and the bound conjugate can be quantitatively detected by reaction with a substrate giving a colorimetric endpoint. The reagents were prepared as follows:
Calibrators 2 to 5 were reconstituted with 1 mL of distilled water, gently agitated and allowed to stand for 15 minutes before use.
Conjugate concentrate (2.5 mL) was diluted using conjugate diluent (10 mL) and mixed gently before use.
1 part Wash Buffer Concentrate (28 mL) was diluted with 14 parts of distilled water (392 mL).

Calibrators 1 to 5, provided in the kit, were used to prepare the standard curve. Heparin plasma was used for this assay and no prior dilution was required. The assay procedure was as follows:
25 μL of calibrator, sample or control was pipetted into the wells;
100 μL of conjugate was added to each well and the plate incubated on a plate shaker at room temperature for 60 minutes;
The plate was aspirated and washed three times with Wash Buffer using a squirty bottle and all remaining wash buffer was removed by inverting the plate on to paper towel;
100 μL of substrate solution was added to each well and the plate was incubated at room temperature on a plate shaker for 10 minutes;
100 μL of stop solution was added to each well and the optical density measured within 30 minutes using a microplate reader (Elisa Tecan Genios, Zurich, Switzerland) set at 450 nm with a reference wavelength at 620 nm.

### 2.5 Vascular Stiffness Quantification

Arterial stiffness was measured using Digital Volume Pulse. It has been shown that the stiffness index (SI_{DVP}) that can be derived from the DVP waveforms is highly correlated to arterial stiffness. The digital volume pulse was recorded by measuring the transmission of infra-red light absorbed through the finger. The amount of light is directly proportional to the volume of blood in the finger pulp. A photoplethysmograph was placed on the index finger of the right hand and waveforms recorded three times over 10 second periods with 5 minute intervals between measurements. The SI_{DVP} is derived from the measured waveform as set forth in FIG. 3 and is obtained from subject height divided by the time between the systolic and diastolic peaks of the DVP. It is a measure of large artery stiffness.

### 2.6 Statistical Analyses

Differences from baseline were used for the statistical analysis. Genotype groups were compared by general linear modelling, including baseline, age, BMI and gender as covariates. A two-sided 5% significance level was used for each endpoint.

### 2.7 Results

The characteristics of the study population are described in table 2. The two genotype groups were balanced for age, BMI, body weight and waist circumference. No significant difference was between genotype groups detected using the t-test.

TABLE 2
Characteristics of the study population (SE = standard error).

| | |
|---|---|
| AA genotype | GG genotype |

| Variable | Mean | SE | Mean | SE |
|---|---|---|---|---|
| Age (years) | 57.8 | 3.81 | 51.4 | 5.05 |
| BMI (Kg/m²) | 27.02 | 0.72 | 27.67 | 0.54 |
| Body weight (Kg) | 83.73 | 3.67 | 83.85 | 2.22 |
| Waist circumference (cm) | 95.72 | 2.19 | 94.75 | 1.13 |

Changes in insulin and SI_{DVP} levels after ingestion of the decaffeinated green tea extract are shown in table 3 for each genotype group.

### 2.8 Discussion

Genotype was found to influence vascular stiffness (SI_{DVP}), with the GG group showing greater improvement after ingestion of the DGT extract than the AA group. There was also a genotype difference in plasma insulin levels, with the GG group displaying greater excursions than the AA group. As postprandial glucose levels did not differ between the groups this observation suggests that the AA group is more insulin sensitive than the GG group.

TABLE 3
Changes in insulin and SI_{DVP} levels after ingestion of the
decaffeinated green tea extract (data shown as change from baseline).

| | | AA Group | | GG Group | | |
|---|---|---|---|---|---|---|
| Measure | Time | Mean | SE | Mean | SE | P-value* |
| Insulin (pmol/L) | 120 | 255.5 | 32.3 | 382.9 | 63.5 | 0.019 |
| | 180 | 216.5 | 30.0 | 373.2 | 59.7 | 0.0079 |
| | 240 | 115.3 | 29.7 | 177.9 | 63.3 | 0.22 |
| | 360 | 188.3 | 30.2 | 194.0 | 39.6 | 0.66 |
| | 480 | 42.4 | 7.8 | 65.4 | 34.1 | 0.43 |
| SI_{DVP} (m/s) | 120 | −0.2 | 0.7 | −2.1 | 0.7 | 0.044 |
| | 240 | 0.6 | 0.7 | −1.5 | 0.7 | 0.026 |
| | 360 | −0.5 | 0.3 | −1.5 | 0.7 | 0.33 |
| | 480 | −0.6 | 0.4 | −1.9 | 0.8 | 0.14 |
| Glucose (mmol/L) | 120 | 2.3 | 0.2 | 2.5 | 0.3 | 0.70 |
| | 180 | 1.0 | 0.4 | 1.0 | 0.4 | 0.99 |
| | 240 | 0.2 | 0.4 | 0.3 | 0.5 | 0.85 |
| | 360 | 0.7 | 0.4 | 0.5 | 0.3 | 0.67 |
| | 480 | 0.0 | 0.2 | 0.0 | 0.2 | 0.78 |

*General linear modelling adjusted for age, BMI, gender and baseline.

### 3. Determining COMT Activity from Protein Fraction Extraction

The activity of COMT can be determined using S-adenosyl-L-methionine as a methyl donor and 3,4-dihydroxybenzoic acid as a substrate as set forth in Syvänen et al, Pharmacogenetics (1997), 7, 65-71 (page 66). The 3-O- and 4-O-methylated reaction products, indicative of enzyme activity, were measured by high performance liquid chromatograph with electrochemical detection.

### 4. Determining COMT Genotype from Urine

COMT genotype can be determined by measuring the level of COMT substrate, methylated catechol-O-methyltransferase substrate, downstream metabolites of methylated catechol-O-methyltransferase substrate or mixtures thereof using analytical techniques known to the person skilled in the art such as high performance liquid chromatography—mass spectrometry. Examples of a suitable COMT substrate, a suitable methylated catechol-O-methyltransferase substrate and a suitable downstream metabolite of methylated catechol-O-methyltransferase substrate are epigallocatechin, O-methylated epigallocatechin and gallic acid (or O-methylated gallic acid) respectively.

1. A method for determining the predisposition of an individual to epigallocatechin gallate, catechin, gallocatechin, catechin gallate, gallocatechin gallate, epicatechin, epigallocatechin, epicatechin gallate and mixtures thereof for the treatment and/or prevention of at least one of:
(a) high diastolic blood pressure;
(b) type II diabetes; and
(c) vascular stiffness;
the method comprising the steps of:
(i) obtaining an ex-vivo sample of an individual;
(j) determining the catechol-O-methyltransferase genotype of the individual from the sample or the catechol-O-methyltransferase activity of the sample;
(k) determining that when the individual has an adenine-adenine or an adenine-guanine catechol-O-methyltransferase genotype or a lowest quartile of catechol-O-methyltransferase activity,
(A) they are more sensitive to treatment by epigallocatechin gallate for the treatment and/or prevention of high diastolic blood pressure than an individual with a guanine-guanine catechol-O-methyltransferase genotype or a highest quartile of catechol-O-methyltransferase activity;
(B) they are more sensitive to treatment by epigallocatechin gallate for the treatment and/or prevention of type II diabetes than an individual with a guanine-guanine catechol-O-methyltransferase genotype or a highest quartile of catechol-O-methyltransferase activity; and
(C) they are less sensitive to treatment by epigallocatechin gallate for the treatment and/or prevention of vascular stiffness than an individual with a guanine-guanine catechol-O-methyltransferase genotype or a highest quartile of catechol-O-methyltransferase activity.

2. A method according to claim 1 wherein the predisposition of an individual is to epigallocatechin gallate.

3. A method according to claim 1 wherein the step of determining the catechol-O-methyltransferase genotype of the individual comprises the step of extracting genomic deoxyribonucleic acid from the sample.

4. A method according to claim 1 wherein the step of determining the catechol-O-methyltransferase activity of the sample comprises the steps of extracting a protein fraction from the sample, the protein fraction comprising catechol-O-methyltransferase, and then contacting the protein fraction with a substrate which would indicate catechol-O-methyltransferase activity.

5. A method according to claim 1 wherein the ex-vivo sample is urine and the step of determining the catechol-O-methyltransferase activity of the sample comprises the step of determining the level of species selected from the group consisting of catechol-O-methyltransferase substrate, methylated catechol-O-methyltransferase substrate, downstream metabolites of methylated catechol-O-methyltransferase substrate and mixtures thereof.
